(19) United States
(12) Patent Application Publication
(10) Pub. No.: US 2019/0142786 A1
(43) Pub. Date: May 16, 2019

(54) METHODS OF TREATING OBESITY IN RESPONDER AND NON-RESPONDER POPULATIONS

(71) Applicant: Vivus, Inc., Campbell, CA (US)

(72) Inventors: Peter TAM, Campbell, CA (US); Roman DVORAK, Campbell, CA (US); Craig PETERSON, Campbell, CA (US)

(21) Appl. No.: 16/007,420

(22) Filed: Jun. 13, 2018

Related U.S. Application Data
(63) Continuation of application No. 14/216,796, filed on Mar. 17, 2014, now abandoned.
(60) Provisional application No. 61/790,991, filed on Mar. 15, 2013.

(51) Int. Cl. A61K 31/35; A61K 9/50; A61K 31/137

(52) U.S. Cl. CPC A61K 31/35; A61K 9/5047; A61K 9/2086; A61K 31/137; A61K 9/5078

(57) ABSTRACT

The disclosed embodiments relate to, dosing regimens for the administration of topiramate, optionally in combination with one or more sympathomimetic agents such as phentermine. The dosing regimens can, for example, limit the exposure of subjects to topiramate, identify subjects who are unlikely to obtain a benefit from treatment with escalating dosages of topiramate (with or without the sympathomimetic agent, such as phentermine), or both, thereby reducing or eliminating harmful or intolerable side effects in subjects who are unlikely to respond to treatment and maximizing the therapeutic benefits from treatment in subjects who do respond.

### CROSS-REFERENCE TO RELATED APPLICATIONS

This application is a continuation of U.S. application Ser. No. 14/216,796, filed Mar. 17, 2014, which claims priority to, and the benefit of, U.S. Provisional Application No. 61/790,991, filed Mar. 15, 2013, the entire contents of each of which are hereby incorporated by reference in their entireties.

### FIELD OF INVENTION

The disclosure relates to, among other things, dosing regimens for the administration of topiramate, optionally in combination with one or more sympathomimetic agents such as phentermine. The dosing regimens can, for example, limit the exposure of subjects to topiramate, identify subjects who are unlikely to obtain a benefit from treatment with escalating dosages of topiramate (with or without the sympathomimetic agent, such as phentermine), or both, thereby reducing or eliminating harmful or intolerable side effects in subjects who are unlikely to respond to treatment and maximizing the therapeutic benefits from treatment in subjects who do respond.

### BACKGROUND OF THE INVENTION

The prevalence of obesity in children and adults is on the rise in many nations, including both developed nations such as the United States and developing nations such as China and India. Obesity can be associated with a variety of medical problems, such as one or more of diabetes, shortness of breath, asthma, pulmonary hypertension, gallbladder disease, dyslipidemia, for example, hyperchloesteremia, dyslipidic hypertension, osteoarthritis, reflux esophagitis, snoring, sleep apnea, menstrual irregularities, infertility, pregnancy complications, gout, coronary artery disease, heart disease, muscular dystrophy, metabolic disorders such as hypoalphalipoproteinemia, familial combined hyperlipidimiea, and Syndrome X, for example, insulin-resistant Syndrome X. Obesity is also associated with increased incidence of some cancers, such as cancers of the colon, rectum, prostate, breast, uterus, and cervix. Obese subjects can also suffer emotional problems related to societal reactions towards obesity.

In addition to being related to incidence of various diseases, obesity can increase the risk of death from hypertension, dyslipidemia, diabetes, such as type II diabetes mellitus, coronary artery disease, heart disease, stroke, gallbladder disease, osteoarthritis, liver disease, and cancers, such as endometrial, breast, prostate, and colon cancers (see, for example, Pi-Sunyer et al. Postgrad Med 2009:121:21-33). Obesity can also be associated with increased all-cause mortality.

Weight loss can mitigate many of the problems associated with obesity, for example, those mentioned above. Behavior modification, such as diets low in fats and overall calories, alone or in combination with increased exercise, can be effective for weight loss. In the long-term, however, weight loss through behavior modification can be difficult to achieve and maintain. As such, pharmaceutical intervention is often appropriate.

Pharmaceuticals useful for treatment of obesity, however, can be associated with harmful side effects. For example, a combination of fenfluramine and phentermine was previously marketed in the United States for treatment of obesity, however, this therapy was removed from the market because of potentially fatal side effects tied to fenfluramine. Furthermore, while many nutritional supplements and herbal remedies are claimed to be useful in effecting weight loss, these claims are often unsubstantiated by rigorous clinical studies.

Topiramate, a sulfamate-substituted monosaccharide with the chemical name 2,3,4,5-bis-O-(1methyletylidene)-β-D-fructopyranose sulfamate, has been reported for use in treating obesity and promoting weight loss, for example, in U.S. Pat. Nos. 7,056,890, 8,580,298, and 8,580,299, and is also marketed for treating migraine headaches and seizure related disorders. A variety of dosages of topiramate can be used for these purposes, depending on the weight, age, gender, and other characteristics of the subject. Although efficacious for these purposes, topiramate is known to have harmful side effects in some subjects. Furthermore, some subjects do not respond to topiramate treatment for obesity. Thus, there is a need for a dosing regimen for topiramate that minimizes subjects' exposure to topiramate while providing one or more indications of whether a particular subject is likely to experience harmful side effects and/or respond to topiramate treatment. The embodiments described herein can meet these and other needs.

### SUMMARY OF THE INVENTION

In one embodiment, a dosing regimen includes measuring the weight of a subject to obtain an initial subject weight, administering a daily dose of about 23 mg of topiramate to the subject for about two weeks after measuring the initial weight of the subject, administering a daily dose of about 46 mg of topiramate to the subject for about three months after the about two weeks of administering the daily dose of about 23 mg of topiramate, measuring the weight of the subject to determine a first weight change from the initial subject weight, and wherein if the weight of the subject has decreased by about 3% or more, maintaining administration of the daily dose of about 46 mg of topiramate; or wherein if the weight of the subject has not decreased by about 3% or more, administering a daily dose of about 69 mg of topiramate. In a further embodiment, the about 69 mg of topiramate can be administered for about two weeks after determining the first weight change. In another embodiment, a daily dose of about 92 mg of topiramate can be administered for about three months after the about two weeks of administering the daily dose of about 69 mg of topiramate.

In some embodiments, a method of administering topiramate includes administering topiramate according to one or more of the above-mentioned embodiments. In other embodiments, a method of administering topiramate includes halting administration of topiramate to a subject who has not lost a specified amount of weight after administration of a daily dose of 46 mg or 92 mg of topiramate for about three months. In particular embodiments, the specified amount of weight is 3% or 5% of the subject's initial body weight.

In an additional embodiment, a method of minimizing exposure to topiramate includes halting administration of topiramate in a subject to whom topiramate has been administered according to all or part of the foregoing embodiments in the event the subject has not lost a specified amount of weight relative to the subjects starting weight prior to the beginning of treatment and optionally in the event the subject is experiencing one or more adverse effects. In another embodiment, the subject to whom topiramate administration is halted has taken a daily dose of about 23 mg of topiramate for about two weeks and a daily dose of about 46 mg of topiramate for about three months. In another embodiment, the subject has not lost at least a specified amount of the initial subject weight after the about three months. In yet another embodiment, the subject in whom topiramate administration is halted has taken a daily dose of about 23 mg of topiramate for about two weeks, a daily dose of about 46 mg of topiramate for about three months, a daily dose of about 69 mg of topiramate for about two weeks, and a daily dose of about 92 mg of topiramate for about three months.

In an additional embodiment, a daily dose of one or more sympathomimetic agents can be administered with one or more of daily dose of about 23 mg topiramate, the daily dose of about 46 mg topiramate, the daily dose of about 69 mg topiramate, and the daily dose of about 92 mg topiramate. In a further embodiment, the sympathomimetic agent can be phentermine. In another embodiment, a daily dose of about 3.75 mg of phentermine can be administered with the daily dose of about 23 mg of topiramate. In yet another embodiment, a daily dose of about 7.5 mg of phentermine can be administered with the daily dose of about 46 mg of topiramate. In still another embodiment, a daily dose of about 11.25 mg phentermine can be administered with the daily dose of about 69 mg of topiramate. In a further embodiment, a daily dose of about 15 mg of phentermine can be administered with the daily dose of about 92 mg of topiramate.

In a particular embodiment, the one or more sympathomimetic agents can be administered in an immediate release form. In a specific embodiment, the topiramate can be administered in a controlled release form. In other embodiments, the controlled release form is a polymer coated bead. In an additional embodiment, the one or more sympathomimetic agents can be administered in an immediate release form and the topiramate can be administered in a controlled release form. In some embodiments, the one or more sympathomimetic agents and the topiramate are administered in a single unit dosage form having a controlled release topiramate portion and an immediate release phentermine portion.

In specific embodiments, a composition can include one or more sustained release topiramate beads that consist essentially of about 1% to about 5% of a binder, such as methyl cellulose, about 40% to about 60% of a filler, such as microcrystalline cellulose, about 25% to about 50% of an active agent, the active agent consisting of topiramate, and a sustained release polymer coating that can consist essentially of ethyl cellulose and polyvinylpyrrolidone and can be present in about 2% to about 10%, such as about 5% or about 5.5% by weight. In some embodiments, the one or more topiramate beads are present with a glidant, such as talc, in a topiramate bead glidant blend.

In other embodiments, the topiramate beads or the topiramate bead glidant blend is present with one or more first phentermine beads that consist essentially of an inert core, which can be a sugar sphere, about 3% to about 6% phentermine on the inert core, and about 2% to about 8% of a binder, which can be hydroxypropyl methylcellullose, on the inert core. In yet other embodiments, the topiramate beads or the topiramate bead glident blend is present with one or more second phentermine beads that consist essentially of an inert core, which can be a sugar sphere, about 6.5% to about 13% of phentermine on the inert core, and about 4% to about 15% of a binder, which can be hydroxypropyl methylcellullose, on the inert core.

In some embodiments, one or more of the foregoing combinations of beads can be present in a tablet or a capsule. In other embodiments, any of the foregoing dosage forms, such as tablets, capsules, mixtures of beads, etc., can be used in any of the foregoing methods or dosing regimens.

### BRIEF DESCRIPTION OF THE DRAWINGS

FIG. 1 is a flow chart illustrating an exemplary dosing regimen for topiramate;

FIG. 2 is a flow chart illustrating an exemplary dosing regimen for topiramate and phentermine;

FIG. 3 is a flow chart illustrating an exemplary dosing regimen for topiramate and phentermine with discontinuation of treatment if weight does not decrease and an adverse side effect is experienced;

FIG. 4 is a chart showing the prevalence of side effects in patients administered placebo, responder patients who lost at least 3% of their initial body weight after administration of topiramate and non-responder patients who did not lose at least 3% of their initial body weight after administration of topiramate and phentermine for three months; and

FIG. 5 is a chart showing the prevalence of side effects in patients administered placebo, responders who lost at least 5% of their initial body weight after administration of topiramate extended-release and phentermine and non-responder patients who did not lose at least 5% of their initial body weight after administration of topiramate extended-release and phentermine for three months.

FIG. 6 is a graph showing percent weight change over time for all observed data in a 2-year cohort for mid-dose phentermine plus topiramate extended-release compared to placebo.

FIG. 7 is a chart showing the effect of combined treatment with either the mid or top dose of the combined dosing regimen of phentermine plus topiramate (extended-release) in comparison to placebo on a selection of risk factors.

### DETAILED DESCRIPTION

In this application, including the appended claims, the singular forms “a,” “an,” and “the” are often used for convenience. However, it should be understood that these singular forms include the plural unless otherwise specified. It should also be understood that all patents, publications, journal articles, technical documents, and the like, referred to in this application, are hereby incorporated by reference in their entirety and for all purposes.

Unless otherwise defined, all terms used in this application should be given their standard and typical meanings in the art, and are used as those terms would be used by a person of ordinary skill in the art at the time of the invention.

“Active agent” as used herein encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds as will be discussed infra. Therefore, reference to “phentermine,” for example, encompasses not only phentermine per se but also salts and other derivatives of phentermine, e.g., phentermine hydrochloride. It is to be understood that when amounts or doses are specified, that those amounts or doses refer to the amount or dose of active agent per se and not to a salt or the like. For example, when it is indicated that a dose or amount of phentermine is 7.5 mg, that would correspond to 9.84 phentermine hydrochloride and not 7.5 phentermine hydrochloride.

“Administering” as used herein includes to any route of administration, for example, oral, parenteral, intramuscular, transdermal, intravenous, inter-arterial, nasal, vaginal, sublingual, subungal, etc. Administering can also include prescribing a drug to be delivered to a subject, for example, according to a particular dosing regimen, or filling a prescription for a drug that was prescribed to be delivered to a subject, for example, according to a particular dosing regimen.

“Body Mass Index” or “BMI” as used herein is an index of weight-for-height that is commonly used to classify overweight and obesity in adults. BMI may be calculated by multiplying an individual's weight, in kilograms, by height, in meters. Currently the CDC and WHO define obesity as having a BMI of 30 or higher. A BMI between 25 and 29.9 is considered overweight. A BMI over 40 is sometimes characterized as morbidly obese. Individuals having a BMI between 30 and 35 may also be referred to as moderately obese, from 35 to 40 severely obese and over 40 very severely obese.

“Halting administration” as used herein includes ceasing to administer, by any route of administration, for example those discussed above. Halting administration can also include, but is not limited to, refusing to provide one or more additional or refill prescriptions, or refusing to fill one or more additional or refill prescription. Halting administration can further include, but is not limited to, a gradual decrease, for example, by decreasing the dosage administered, frequency of administration, or both, over a period of time until the patient is no longer administered the drug. For example, taking a dose every other day for 1-2 weeks before stopping treatment altogether.

A “daily dose” of a particular material refers the amount of the material administered in a day. A daily dose can be administered as a single dose or as multiple doses. When a daily dose is administered as multiple doses, the daily dose is the sum of the amount of material administered in all of the multiple doses that are administered over the course of one day. For example, a daily dose of 12 mg can be administered in a single 12 mg dose once per day, in 6 mg doses administered twice per day, in 4 mg doses administered three times per day, in 2 mg doses administered six times per day, etc. The multiple doses can be the same or different doses of the material, unless otherwise specified. When a daily dose is administered as multiple doses, the multiple doses can be administered by the same or different route of administration, unless otherwise specified. Thus, a daily dose of 12 mg can include, for example, a 10 mg intramuscular dose and a 2 mg oral dose administered over the course of one day.

The term “dosage form” denotes any form of a pharmaceutical composition that contains an amount of active agent sufficient to achieve a therapeutic effect with a single administration. When the formulation is a tablet or capsule, the dosage form is usually one such tablet or capsule, although this is not required unless otherwise specified. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release, modified release and delayed release formulations.

Administration of one compound “with” a second compound, as used herein, includes but is not limited to cases where the two compounds are administered simultaneously or substantially simultaneously. For example, administration of a first compound with a second compound can include administering the first compound in the morning and administering the second compound in the evening, as well as administering the first and second compounds in the same dosage form or in two different dosage forms that at the same or nearly the same time.

“Topiramate” as used herein includes not only the chemical compound 2,3,4,5-bis-O-(1methyletylidene)-β-D-fructopyranose sulfamate, but also all stereoisomers, such as enantiomers and diasteriomers, thereof, as well as salts, mixed salts, polymorphs, solvates, including mixed hydrates and mixed solvates, of one or more stereoisomers or mixtures of stereoisomers.

“Phentermine” as used herein includes not only the chemical compound 2-methyl-1-phenylpropan-2-amine, but also all stereoisomers, such as enantiomers and diasteriomers, thereof, as well as salts, mixed salts, polymorphs, solvates, including mixed hydrates and mixed solvates, of one or more stereoisomers or mixtures of stereoisomers.

A “subject” or multiple “subjects” can be members of any species, typically human. The subjects of all experiments and studies discussed herein were human except when otherwise indicated.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient provides a measurable time delay before drug is released from the formulation into the patient's body.

The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of topiramate, such that the unit dosage in each tablet or capsule is approximately 50% of the therapeutically effective amount.

Topiramate can be administered to effect weight loss in a subject, however, potentially harmful side effects of topiramate therapy are known. Further, some subjects do not lose weight in response to topiramate therapy. For those subjects, the dosage of topiramate can be increased. Such an increase in dosage can also increase the likelihood of potentially harmful side effects. Thus, a dosing regime is provided that can identify subjects who are likely to have potentially harmful side effects before the maximum dosage of topiramate is reached.

The Food and Drug Administration (FDA) deems a pharmaceutical weight loss treatment to have a clinical effect on a subject only if the subject loses 5% or more of his or her body mass over a time period of about six months. The inventors have made the surprising and unexpected discovery that subjects who exhibit a body mass loss that is less than the clinically effective level, as defined by the FDA, after treatment with the specific dosing regimens defined herein are significantly more likely than others to experience harmful side effects and are less likely to achieve the desired benefits of the treatment. Such subjects may be identified as non-responders after about two weeks to about six months, for example about one month to about six months, about two months to about five months, or about 3.5 months of treatment and treatment may be halted.

The dosing regimen can include measuring the weight of a subject to obtain an initial subject weight. The initial subject weight can be obtained before administration of topiramate. Topiramate can then be administered to the subject at a first daily dose, which can be an initial dose that can, but need not be, therapeutically effective. The initial daily dose of topiramate can be, for example, about 5 mg to about 60 mg, about 10 to about 50 mg, about 15 mg to about 30 mg, or about 23 mg. This initial daily dose of topiramate can be administered for a first time period, which can be, for example, about two weeks, or for about two days to about two weeks, about one week to about two weeks, or about one week to about one month, after measuring the initial subject weight.

After the first time period of administering the initial daily dose, a second daily dose of topiramate that is higher than the initial daily dose can be administered. The second daily dose can, for example, be about double any of the initial daily doses or initial daily dose ranges discussed above, for example, about 46 mg of topiramate, or about 10 mg to about 75 mg or about 35 mg to about 55 mg. The second daily dose of topiramate can be administered for a second time period after the first time period, for example, for about three months, about two weeks to about six months or about two months to about four months.

After administration of the second daily dose of topiramate for the second time period, the weight of the subject can be measured again to determine a first weight change of the subject from the initial weight. If, at this point in the dosing regimen, the subject weight has not decreased by, for example, about 3% or more (that is, the subject has a body weight of greater than about 97% of the initial subject body weight), or about 5% or more (that is, the subject has a body weight greater than about 95% of the initial subject body weight), then the subject can be at a higher risk of developing harmful side effects and may be a non-responder. Thus, administration of the topiramate can be halted. On the other hand, subjects whose weight has decreased by, for example, about 1% to about 8% or more, for example, about 3% or more or about 5% or more, may have only a typical or reduced risk of developing harmful side effects compared to all patients. For such patients, administration of the second daily dose, such as about 46 mg, of topiramate can be maintained, for example, for 3, 6, 9, 12, 18, 24 or 36 months or more.

For some patients, for example, those who lose nearly 3% or nearly 5% of their initial body weight after administration of the second daily dose, such as about 46 mg, of topiramate, or those for whom the benefit of additional topiramate therapy outweighs the risk of side effects, a daily dose of topiramate can be increased to a third daily dose. The third daily dose can be higher than the second daily dose, for example, about one-and-a-half times any of the second daily doses or ranges of second daily doses discussed above, from about 40 mg to about 90 mg, or from about 55 mg to about 75 mg, such as about 69 mg, of topiramate. The third daily dose of topiramate can be administered for a third time period, which can be, for example, for about two weeks, for about two days to about two weeks, about two weeks to about 4 weeks, about one week to about one month, after measuring the second time period.

Next, a fourth daily dose of topiramate, which can be higher than the third daily dose, can be administered. The fourth daily dose may be, for example, about double any of the second daily doses or ranges of second daily doses discussed above, about 60 mg to about 110 mg, or about 75 mg to about 100 mg, about 80 mg to about 150 mg, about 92 mg, and can be administered for about two weeks to about six months or two months to about four months, about three months, or about three months to about twelve months, after the third time period.

The fourth daily dose of topiramate can be the maximum daily dose administered in the dosing regimen. Thus, after administration of the fourth daily dose of topiramate for about three months, the weight of the subject can be measured to determine a second weight change of the subject from the initial subject weight. If the subject has not lost a sufficient amount of body weight, for example, about 3% to about 10% or more, such as about 5% or more, of the body weight of the subject based on the initial subject weight, then the subject may not respond to topiramate therapy. Further, the subject can be at greater risk for potentially harmful side effects. Thus, topiramate administration can be halted for a subject who has not lost, for example, about 3% to about 10% or more, or about 5% or more, of the body weight of the subject after the administration of the fourth daily dose of topiramate according to the dosing regimen described herein. Alternatively, administration of the fourth daily dose of topiramate can be maintained for a subject who has lost, for example, about 3% to about 10% or more, such as about 5% or more, of the body weight of the subject after the administration of the fourth daily dose of topiramate. The fourth daily dose may be continued for an additional period of time, for example, for 3, 6, 9, 12, 18, 24 or 36 additional months or more. In some aspects, a patient that is taking the fourth daily dose of topiramate for a period of time may reduce the daily dose after a period of time, for example, after three to six months, and then continue to take a lower daily does for another period of time, for example, six months to two years or longer in order to maintain achieved weight loss.

One or more sympathomimetic agents can be administered concurrently with the administration of topiramate. The one or more sympathomimetic agents can include, for example, one or more of the sympathomimetic agents mentioned in Table 1 of U.S. Pat. No. 8,580,299, which is hereby incorporated by reference in its entirety for all purposes.

One particular sympathomimetic agent that can be administered concurrently with the administration of topiramate is phentermine. The daily dose of the sympathomimetic agent, for example, phentermine, can be any appropriate daily dose. For example, the daily dose of the sympathomimetic agent, for example, phentermine, can be from about 2 mg to about 1,500 mg, for example, a daily dose of about 2 mg to about 20 mg. The daily dose of the sympathomimetic agent can be increased if and when the daily dose of topiramate is increased, although this is not required unless otherwise specified. The ratio of topiramate to phentermine in the different daily doses may be constant, for example, if the first daily dose is 23 mg of topiramate and 3.75 mg phentermine, for a weight of phentermine to topiramate ratio of about 16% (i.e. the weight of the phentermine is about 16% of the weight of the topiramate), then one or more of the second, third, and fourth daily doses can also have about a 16% weight ratio of phentermine to topiramate. Other ratios may also be used, for example, about 10-20%, about 13-17%. The ratio may be maintained for one or more of the second, third and fourth doses. For example, the second daily dose may be about 7.5 mg phentermine and 46 mg topiramate, the third may be about 11.25 mg phentermine and about 69 mg topiramate and the fourth about 15 mg phentermine and about 92 mg topiramate, each daily dose having a ratio of about 16% (the weight of phentermine being about 16% of the weight of phentermine).

Patients can be selected for the treatment regimen disclosed herein based on BMI with or without the presence of weight related comorbidities. Patients having a BMI of 30 or greater or patients having a BMI of 27 or greater with at least one comorbidity such as hypertension, type 2 diabetes mellitus, or dyslipidemia may be selected as candidates for treatment.

A daily dose of 7.5 mg of phentermine can be administered with the daily dose of about 46 mg of topiramate to a patient having a BMI≥35, for about 6 months (optionally after titration up from a low dose, such as after administration of a daily dose of about 23 mg topiramate with about 3.75 mg phentermine for about 2 weeks) and the individual is assessed. If the individual is responding but the BMI is still ≥35 the individual's dose may be elevated to a daily dose of about 15 mg of phentermine in combination with the daily dose of about 92 mg of topiramate (optionally with a 2 week titration of a daily dose of 11.25 mg of phentermine in combination with about 69 mg of topiramate).

Subjects who are candidates to maintain either the daily dose of 46 mg topiramate with 7.5 mg phentermine or the daily dose of 92 mg topiramate with 15 mg phentermine can maintain that regimen in order to manage chronic obesity or being chronically overweight. The combination of low doses of phentermine plus controlled-release topiramate has been demonstrated to be a safe and effective treatment for at least 108 weeks (Garvey et al. Am J Clin Nutr 2011). Subjects who respond to the 7.5 mg phentermine/46 mg topiramate daily dose may continue taking that daily dose after the 3 month period for an additional period of time, for example, for 3, 6, 9, 12, 18, 24, or 36 additional months or more. For some subjects treatment may continue for longer periods, for example, 4 to 6 years, 6 to 10 years, or longer.

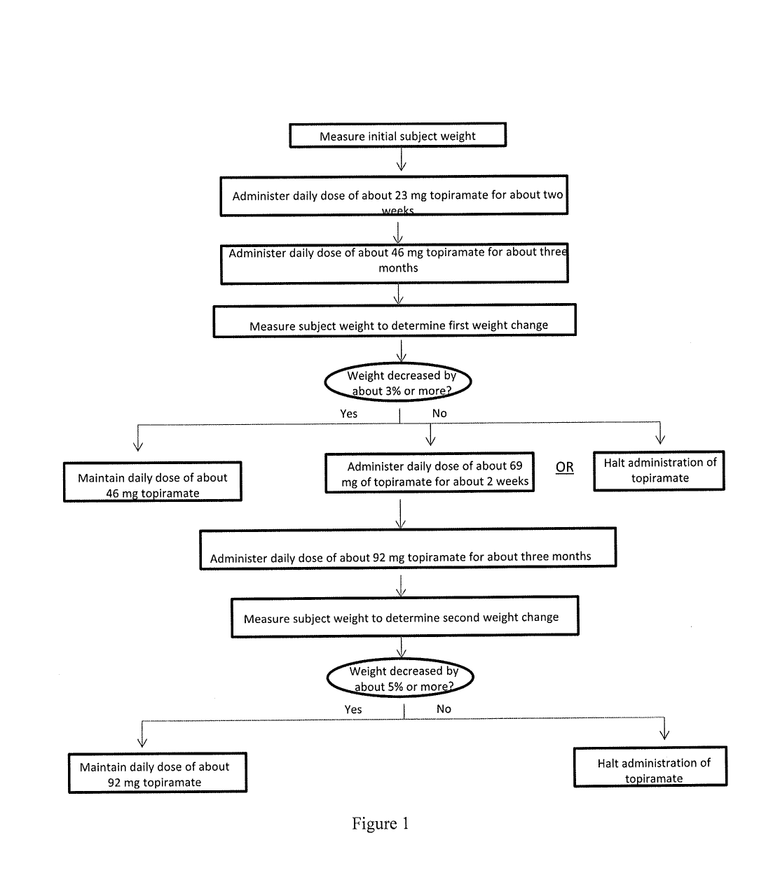

FIG. 1 illustrates an exemplary dosing regimen for administering topiramate to a subject. In FIG. 1, an initial daily dose of about 23 mg of topiramate is administered for about two weeks after an initial subject weight is obtained. Then a second daily dose of about 46 mg of topiramate is administered for about three months after the about two weeks of administering the daily dose of about 23 mg of topiramate. After the about three months of administering a daily dose of about 46 mg of topiramate, the subject weight is measured to determine a first weight change of the subject from the initial weight. If the subject weight decreased by about 3% or more, then administration of a daily dose of about 46 mg of topiramate is maintained. If the subject weight did not decrease by about 3% or more, then either the administration of topiramate is halted or a daily dose of about 69 mg of topiramate is administered for about two weeks.

In FIG. 1, when a daily dose of 69 mg of topiramate is administered for about two weeks as described above, a daily dose of about 92 mg topiramate can be administered for about three months after the about two weeks of administering the daily dose of 69 mg of topiramate. The subject weight can then be measured to determine a second subject weight change from the initial subject weight. If the weight of the subject has decreased by about 5% or more, then the administration of a daily dose of about 92 mg of topiramate can be continued, otherwise administration of topiramate can be halted.

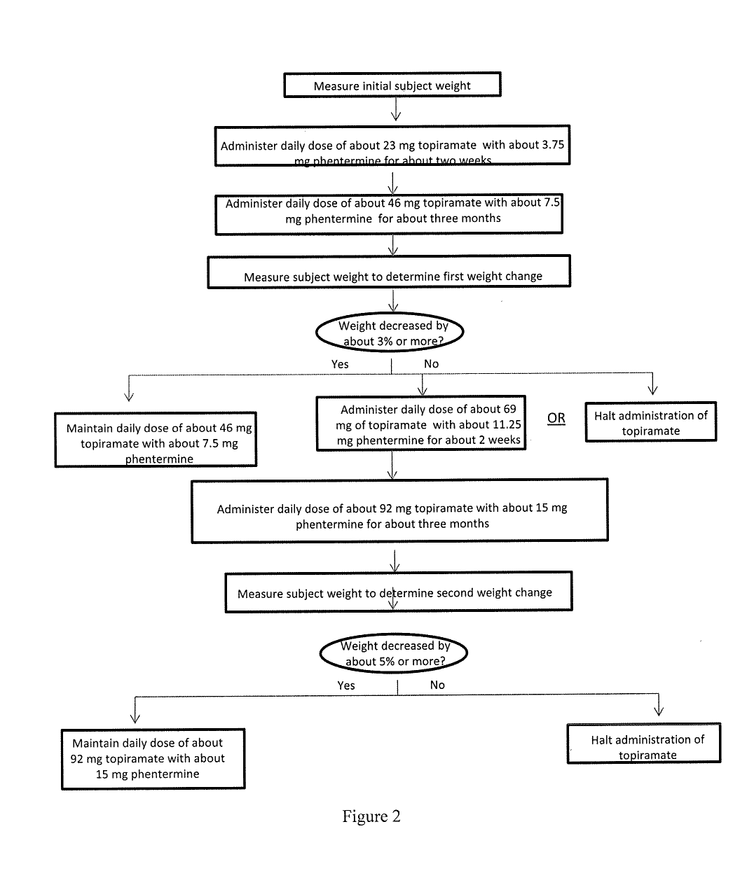

FIG. 2 illustrates an exemplary dosing regimen for administering topiramate with phentermine. In FIG. 2, the doses of topiramate are identical to those discussed and illustrated with respect to FIG. 1, in addition, a daily dose of phentermine is administered with each daily dose of topiramate. In FIG. 2, the daily dose of phentermine is increased when the daily dose of topiramate is increased. Thus, as shown in FIG. 2, a daily dose of about 3.75 mg of phentermine is administered with the daily dose of about 23 mg of topiramate, a daily dose of about 7.5 mg of phentermine is administered with the daily dose of about 46 mg of topiramate, a daily dose of about 11.25 mg of phentermine is administered with the daily dose of about 69 mg of topiramate, and a daily dose of about 15 mg of phentermine is administered with the daily dose of about 92 mg of topiramate.

It should be understood that FIGS. 1 and 2 and the descriptions thereof illustrate examples of dosing regimens and are not intended to be limiting unless otherwise specified.

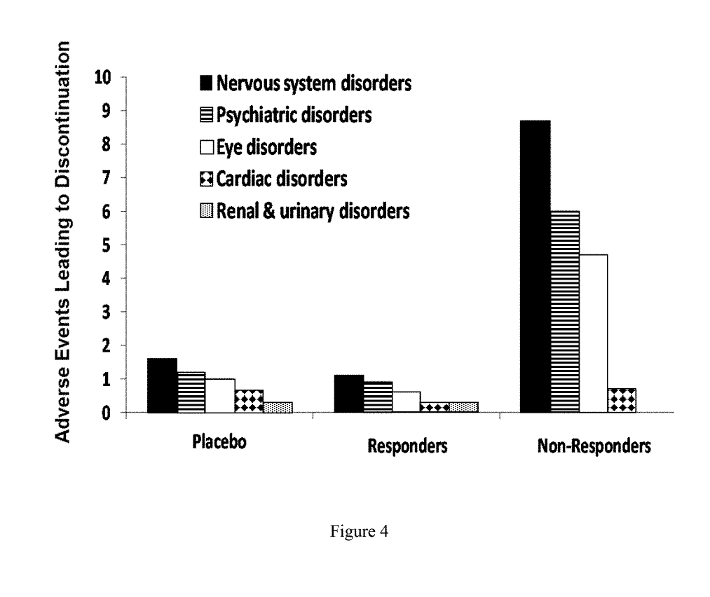

FIG. 4 is a graph showing the prevalence and nature of adverse effects on subjects in a clinical study that were severe enough to warrant discontinuing the subject's participation in the study. The left-hand portion of the graph shows adverse events in subjects on a placebo. The middle column shows adverse events in responder subjects who lost 3% or more of their initial weight after three months of administration of 46 mg topiramate with 7.5 mg phentermine. The right-hand column shows adverse events in non-responder subjects who did not lose 3% or more of their initial weight after three months of administration of 46 mg topiramate with 7.5 mg phentermine.

FIG. 4 shows that the prevalence of all adverse events in responder subjects who lost 3% or more of their initial weight after three months of administration of a daily dose of 46 mg topiramate with 7.5 mg phentermine is essentially identical to that in subjects who took a placebo. However, the prevalence of adverse events in non-responder subjects who did not lose 3% or more of their initial weight after three months administration of a daily dose of 46 mg topiramate with 7.5 mg was significantly higher than in subjects who took a placebo.

The prevalence of adverse events in non-responder subjects who did not lose 3% or more of their initial weight after three months administration of 46 mg topiramate with 7.5 mg is significantly greater than in both responder subjects and in placebo subjects. This is important, because the placebo subjects, like the non-responder subjects, did not lose a significant amount of initial body weight. This shows that the increase in adverse effects is related to the administration of topiramate with phentermine, and is not merely related to the lack of improvement of the obese state of the non-responder subjects.

Furthermore, the prevalence of adverse events effecting each organ system was greater in non-responder subjects who did not lose 3% or more of their initial weight after three months administration of a daily dose of 46 mg topiramate with 7.5 mg than in responder subjects who did. For example, adverse effects affecting the nervous system caused about two percent of placebo subjects and about one percent of responder subjects who lost 3% or more of their initial weight after three months administration of a daily dose of 46 mg topiramate with 7.5 mg to withdraw from the study. For non-responder patients who did not lose 3% or more of their initial weight after three months administration of a daily dose of 46 mg topiramate with 7.5 mg, the withdrawal rate due to adverse effects related to the nervous system was about nine percent of the total number of non-responders, which is about four-and-a-half times higher than for placebo patients or responder patients. Similarly, withdrawal rates for adverse effects related to psychiatric disorders and eye disorders were each substantially higher for non-responder subjects than for placebo subjects or responder subjects (6% compared to about 1% and 4.7% compared to less than 1%, respectively).

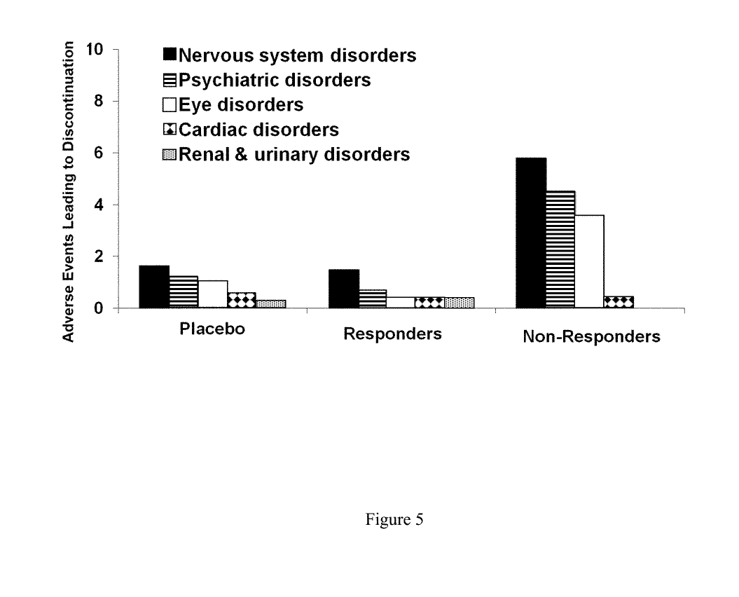

FIG. 5 is a graph showing the prevalence and nature of adverse events on subjects in a clinical study that were severe enough to warrant discontinuing the subjects' participation in the study prior to the end of the 56 weeks of the study. The left-hand portion of the graph shows adverse events in subjects on a placebo as a percentage of the total number of patients in each subgroup. The middle column shows adverse events in responder subjects who lost 5% or more of their initial weight after three months of administration of a daily dose of 46 mg topiramate with 7.5 mg phentermine. The right-hand column shows adverse events in non-responder subjects who did not lose 5% or more of their initial weight after three months of administration of a daily dose of 46 mg topiramate with 7.5 mg phentermine.

FIG. 5 shows that the prevalence of all adverse events in responder subjects who lost 5% or more of their initial weight after three months of administration of a daily dose of 46 mg topiramate with 7.5 mg phentermine is essentially identical to that in subjects who took a placebo.

The prevalence of adverse events in non-responder subjects who did not lose 5% or more of their initial weight after three months administration of 46 mg topiramate with 7.5 mg is significantly greater than in both responder subjects and in placebo subjects. This is important, because the placebo subjects, like the non-responder subjects, did not lose a significant amount of initial body weight. This shows that the increase in adverse effects is related to the administration of topiramate with phentermine, and is not merely related to the lack of improvement of the obese state of the non-responder subjects.

Furthermore, the prevalence of adverse events affecting each organ system was greater in non-responder subjects who did not lose 5% or more of their initial weight after three months administration of a daily dose of 46 mg topiramate with 7.5 mg than in responder subjects who did. For example, adverse effects affecting the nervous system caused 1.6% of placebo subjects and 1.5% of responder subjects who lost 5% or more of their initial weight after three months administration of a daily dose of 46 mg topiramate with 7.5 mg to withdraw from the study. For non-responder subjects who did not lose 5% or more of their initial weight after three months administration of a daily dose of 46 mg topiramate with 7.5 mg, the withdrawal rate due to adverse effects related to the nervous system was almost 6%, which is about four times higher than responder subjects. Withdrawal rates for adverse effects related to psychiatric disorders and eye disorders were both substantially higher for non-responder subjects than for placebo subjects or responder subjects (6.4 fold and 9 fold respectively).

Thus, halting administration of topiramate rather than increasing the dosage can significantly decrease the incidence of adverse events in subjects who do not respond to topiramate treatment, for example, by losing at least 3% or at least 5% of their initial weight after administration of a daily dose of 46 mg topiramate over three months. This result is completely unexpected, since in the pharmaceutical and medical arts subjects who do not respond to a low dose of a pharmaceutical typically increase the dosage until the maximum dose is reached.

Adverse reactions that have been observed with the treatment include the following, grouped by system organ class: (i) Nervous System: paraesthesia, headache, dizziness, dysgeusia, hypoesthesia, disturbance in attention; (ii) Psychiatric Disorders: insomnia, depression anxiety; (iii) gastrointestinal disorders: constipation, dry mouth, nausea, diarrhea, dyspepsia, gastroesophageal reflux disease, paraesthesia oral; (iv) General disorders and administration site conditions: fatigue, irritability, thirst, chest discomfort; (v) eye disorders: vision blurred, eye pain, dry eye; (vi) cardiac disorders: palpitations; (vii) skin and subcutaneous tissue disorders: rash, alopecia; (viii) metabolism and nutrition disorders: hypokalemia, decreased appetite; (ix) reproductive system and breast disorders: dysmenorrheal; (x) infections and infestations: upper respiratory tract infection, nasophyaryngitis, sinusitis, bronchitis, influenza, urinary tract infection, gastroenteritis; (xi) musculoskeletal and connective tissue disorders: back pain, pain in extremity, muscle spasms, musculoskeletal pain neck pain; (xii) respiratory, thoracic and mediastinal disorders: cough, sinus congestion, pharyngoloarygeal pain, nasal congestion. Of these adverse reactions the most commonly observed in treated subjects were paraesthesia, constipation, dry mouth, dysgeusia, dizziness, hypoesthesia, disturbance in attention, irritability and alopecia.

Table 1 shows the adverse reactions reported in greater than or equal to 2% of patients and more frequently than placebo during 1 year of treatment for the overall study population.

TABLE 1

| | | 3.75 mg | 7.5 mg | 15 mg |
|---|---|---|---|---|
| | | phentermine/ | phentermine/ | phentermine/ |
| | | 23 mg | 46 mg | 92 mg |
| System Organ Class | Placebo | topiramate | topiramate | topiramate |
| Preferred Term | (N = 1561) % | (N = 240) % | (N = 498) % | (N = 1580) % |
| Nervous System Disorders | | | | |
| Paraesthesia | 1.9 | 4.2 | 13.7 | 19.9 |
| Headache | 9.3 | 10.4 | 7.0 | 10.6 |
| Dizziness | 3.4 | 2.9 | 7.2 | 8.6 |
| Dysgeusia | 1.1 | 1.3 | 7.4 | 9.4 |
| Hypoesthesia | 1.2 | 0.8 | 3.6 | 3.7 |
| Disturbance in Attention | 0.6 | 0.4 | 2.0 | 3.5 |
| Psychiatric Disorders | | | | |
| Insomnia | 4.7 | 5.0 | 5.8 | 9.4 |
| Depression | 2.2 | 3.3 | 2.8 | 4.3 |
| Anxiety | 1.9 | 2.9 | 1.8 | 4.1 |
| Gastrointestinal Disorders | | | | |
| Constipation | 6.1 | 7.9 | 15.1 | 16.1 |
| Dry Mouth | 2.8 | 6.7 | 13.5 | 19.1 |
| Nausea | 4.4 | 5.8 | 3.6 | 7.2 |
| Diarrhea | 4.9 | 5.0 | 6.4 | 5.6 |
| Dyspepsia | 1.7 | 2.1 | 2.2 | 2.8 |
| Gastroesophageal Reflux Disease | 1.3 | 0.8 | 3.2 | 2.6 |
| Paraesthesia Oral | 0.3 | 0.4 | 0.6 | 2.2 |
| General Disorders and | | | | |
| Administration Site Conditions | | | | |
| Fatigue | 4.3 | 5.0 | 4.4 | 5.9 |
| Irritability | 0.7 | 1.7 | 2.6 | 3.7 |
| Thirst | 0.7 | 2.1 | 1.8 | 2.0 |
| Chest Discomfort | 0.4 | 2.1 | 0.2 | 0.9 |
| Eye Disorders | | | | |
| Vision Blurred | 3.5 | 6.3 | 4.0 | 5.4 |
| Eye Pain | 1.4 | 2.1 | 2.2 | 2.2 |
| Dry Eye | 0.8 | 0.8 | 1.4 | 2.5 |
| Cardiac Disorders | | | | |
| Palpitations | 0.8 | 0.8 | 2.4 | 1.7 |
| Skin and Subcutaneous | | | | |
| Tissue Disorders | | | | |
| Rash | 2.2 | 1.7 | 2.0 | 2.6 |
| Alopecia | 0.7 | 2.1 | 2.6 | 3.7 |
| Metabolism and | | | | |
| Nutrition Disorders | | | | |
| Hypokalemia | 0.4 | 0.4 | 1.4 | 2.5 |
| Decreased Appetite | 0.6 | 2.1 | 1.8 | 1.5 |
| Reproductive System and | | | | |
| Breast Disorders | | | | |
| Dysmenorrhea | 0.2 | 2.1 | 0.4 | 0.8 |
| Infections and Infestations | | | | |
| Upper Respiratory Tract Infection | 12.8 | 15.8 | 12.2 | 13.5 |
| Nasopharyngitis | 8.0 | 12.5 | 10.6 | 9.4 |
| Sinusitis | 6.3 | 7.5 | 6.8 | 7.8 |
| Bronchitis | 4.2 | 6.7 | 4.4 | 5.4 |
| Influenza | 4.4 | 7.5 | 4.6 | 4.4 |
| Urinary Tract Infection | 3.6 | 3.3 | 5.2 | 5.2 |
| Gastroenteritis | 2.2 | 0.8 | 2.2 | 2.5 |
| Musculoskeletal and | | | | |
| Connective Tissue Disorders | | | | |
| Back Pain | 5.1 | 5.4 | 5.6 | 6.6 |
| Pain in Extremity | 2.8 | 2.1 | 3.0 | 3.0 |
| Muscle Spasms | 2.2 | 2.9 | 2.8 | 2.9 |
| Musculoskeletal Pain | 1.2 | 0.8 | 3.0 | 1.6 |
| Neck Pain | 1.3 | 1.3 | 2.2 | 1.2 |
| Respiratory, Thoracic, and | | | | |
| Mediastinal Disorders | | | | |
| Cough | 3.5 | 3.3 | 3.8 | 4.8 |
| Sinus Congestion | 2.0 | 2.5 | 2.6 | 2.0 |
| Pharyngolaryngeal Pain | 2.0 | 2.5 | 1.2 | 2.3 |
| Nasal Congestion | 1.4 | 1.7 | 1.2 | 2.0 |
| Injury, Poisoning, and | | | | |
| Procedural Complications | | | | |
| Procedural Pain | 1.7 | 2.1 | 2.4 | 1.9 |

Close inspection of FIG. 4 and FIG. 5 shows that the prevalence of harmful side effects in non-responders who did not lose about 3% or more of their initial weight after administration of a daily dose of 46 mg topiramate over three months is significantly greater than in non-responders who did not lose about 5% or more of their initial weight. For example, about 50% more non-responders who did not lose about 3% or more of their initial weight (FIG. 4) had central nervous system side effects leading to study withdrawal than the non-responders who did not lose 5% or more of their initial weight (FIG. 5). This result is particularly surprising because a loss of 3% of an initial subject weight is not clinically significant. Thus, prior to this invention, a person of ordinary in the art would have believed that that a subject who has been treated with topiramate, such as a daily dose of 46 mg of topiramate, for only about three months but has not lost at least 3% of the initial subject body weight should either be allowed to complete the six month time period allowed by the FDA or be administered an increased daily dose of topiramate. However, the inventors have shown that halting the administration of topiramate before the six month period and before administering a higher dose of topiramate can advantageously reduce the likelihood of harmful side effects.

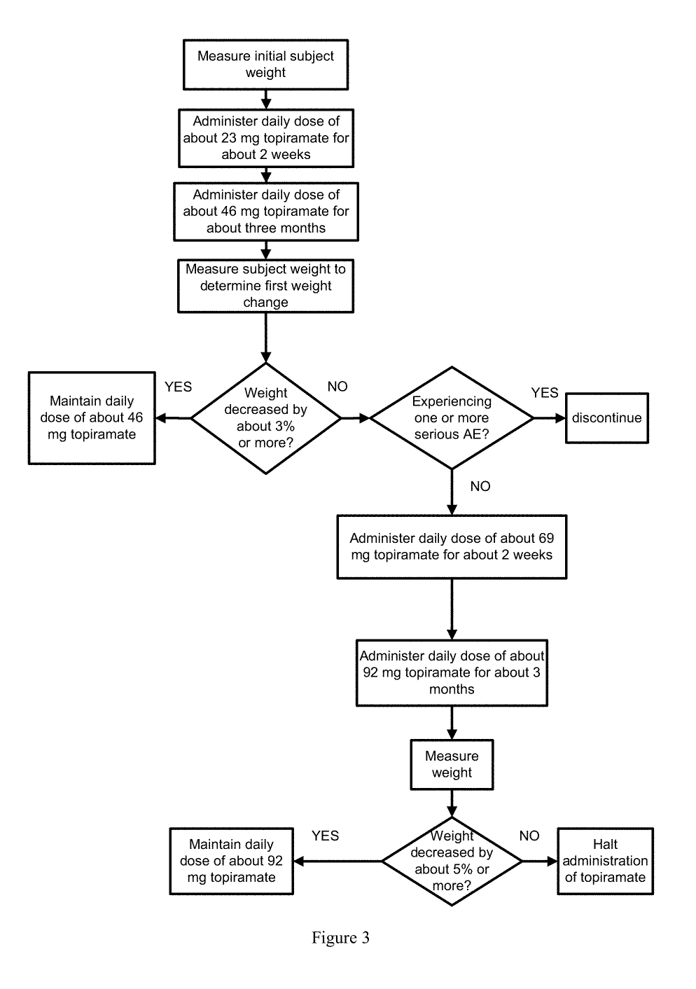

In another aspect, treatment may be discontinued if the subject has both failed to achieve at least 3% weight loss after 3 months of treatment and has or is experiencing during treatment at least one adverse effect. An example of this method is illustrated in FIG. 3. After determining that the patient has not achieved the desired weight loss a determination is made as to whether or not the patient is experiencing a side effect (e.g. and adverse effect or AE). If they are experiencing an adverse effect then treatment is discontinued, if not treatment can be ramped up to the higher dose as shown. In some aspects the assessment is made prior to 3 months of treatment on the 46 mg topiramate dose, for example, a provider may review weight loss at 4, 6, 8 or 10 weeks on that dose and determine if the patient has lost the 3%. Similarly with patients that are taking the 92 mg dose they may be evaluated for 5% weight loss after 4, 6, 8 or 10 weeks. In many non-responders it is possible to determine that he or she is a non-responder after that shortened period of time, particularly in conjunction with the observation of one or more adverse effects.

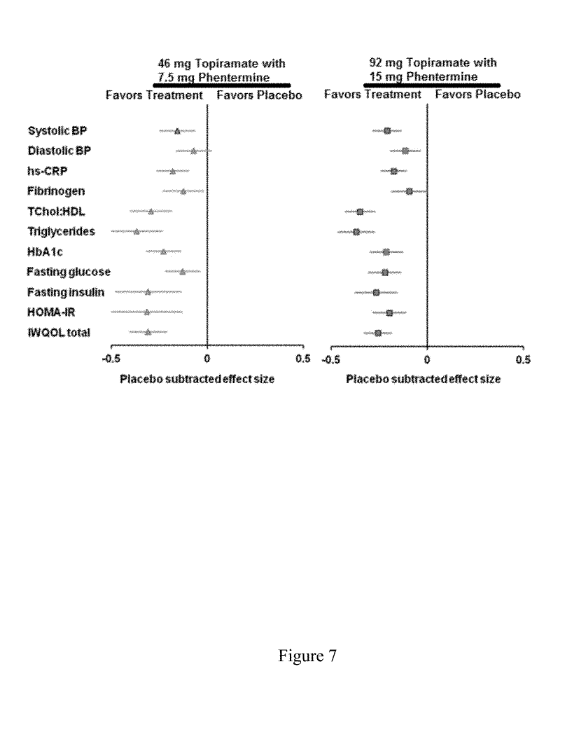

In addition to the unexpected results described with respect to predicting and decreasing side effects, the dosing regimens described herein provide measureable clinical benefits. Table 2 compares the change from baseline to week 56 in weight, systolic blood pressure (SBP), high-density lipoprotein (HDL), triglycerides, fasting insulin, and C-reactive protein (CRP) in placebo subjects, all subjects administered a daily dose of 46 mg topiramate with 7.5 mg phentermine (“All”), and responder subjects who lost either at least 3% or at least 5% of their initial body weight after three months of administration of a daily dose of 46 mg topiramate with 7.5 mg phentermine. Table 2 shows that, in addition to losing more weight than the treated (i.e., non-placebo) subjects as a whole, responder subjects had a significantly larger decrease in SBP, triglycerides, and fasting insulin than the treated subjects as a whole. Further, responder subjects have a greater increase in HDL than treated subjects as a whole. FIG. 7 shows that for a number of clinically relevant measurements, treatment with a combination of topiramate and phentermine results in a significant improvement. Table 2 also shows that non-responders see significantly smaller benefit in weight loss, decrease in SBP, triglycerides and fasting insulin than the 3% responder group or the 5% responder group. Thus, as demonstrated in Table 2, the dosing regimen described herein not only serves to predict side-effects, but also provides a better result to the pool of subjects who remained on topiramate treatment.

TABLE 2

| | | 46 mg Topiramate with 7.5 mg Phentermine | | | |
|---|---|---|---|---|---|
| | Placebo | | Responder | Responder | Non- |
| | All | All | 5% | 3% | Responder |
| | N = 979 | N = 488 | N = 275 | N = 348 | N = 213 |
| % Weight Loss | 1.2 | 7.8 | 12.9 | 12.1 | 3.4 |
| Δ SBP (mmHg) | −2.4 | −4.7 | −6.4 | −6.1 | −2.6 |
| Δ HDL (%) | 1.2 | 5.2 | 7.8 | 7.2 | 1.7 |
| Δ Triglycerides | 4.7 | −8.6 | −16.3 | −15.2 | −2.7 |
| (%) | | | | | |
| Δ Fasting Insulin | 0.7 | −3.5 | −4.8 | −4.5 | −2.2 |
| (ulU/ml) | | | | | |
| Δ CRP (mg/L) | −0.8 | −2.5 | −2.9 | −2.7 | −2.6 |

Close inspection of Table 2 reveals additional surprising and unexpected benefits of the dosing regimens described herein. Specifically, responders who lost 3% or more of their initial weight after administration of a daily dose of 46 mg of topiramate for three months obtained essentially the same benefit in terms of overall weight loss, decreased SBP, triglycerides, and fasting insulin, and increased HDL, as responders who lost 5% or more of their initial weight in the first three months of treatment.

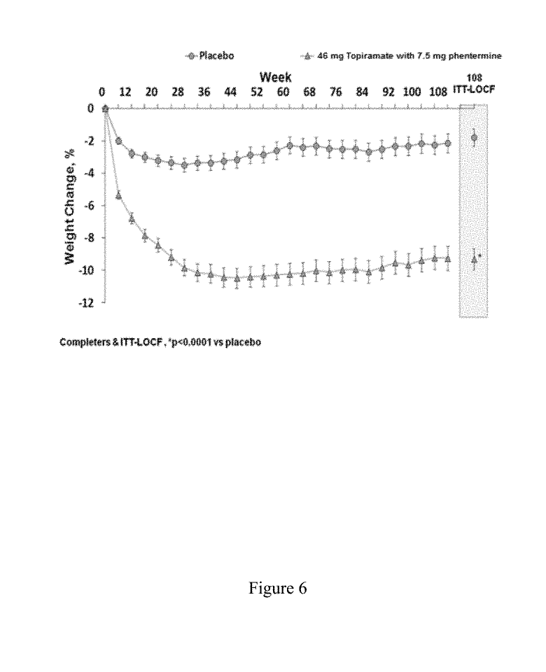

Taken together with the data presented in FIG. 4 and FIG. 5, Table 2 demonstrates another surprising and unexpected feature of the dosing regimens described herein. Specifically, halting administration of topiramate for non-responders according to the dosing regimens described herein permits identification of subjects likely to experience harmful side effects before the six-month time period specified by the FDA for pharmaceutical weight loss therapies to be effective. This early identification can allow minimizing topiramate exposure to subjects who are likely to experience such harmful side effects and unlikely to respond to topiramate treatment. Also, surprisingly, despite such early identification, the dosing regimens described herein are still highly effective at achieving health benefits, such as weight loss, in both responder subjects and in the patient pool as a whole. FIG. 6 shows additional weight loss achieved by administering a daily dose of 46 mg topiramate with 7.5 mg phentermine for 56 weeks.

FIG. 6 shows that subjects using the dosing regimens disclosed herein can continue on the daily dose of 46 mg topiramate (extended release) with 7.5 mg phentermine for an additional 3, 6, 9, 12, 18, or 21 months, for a total of no less than about 24 months of treatment and sustain the observed weight loss.

The dosing regimens described herein, for example those illustrated in FIGS. 1, 2 and 3, can be used in a variety of different methods. One such method is a method of administering topiramate to a subject. In such a method, topiramate can be administered according to the dosing regimens described above, for example, the dosing regimens illustrated in FIG. 1 or FIG. 2. As a variation, only part of the dosing regimens discussed above can be used. For example, if the administration of topiramate is halted after determining the first weight change, then the third and fourth daily doses of topiramate, for example, 69 mg and 92 mg, respectively, need not be administered. Similarly, if the administration of topiramate is initially maintained after the first weight change, the daily dose of topiramate can later be increased.

The dosing regimens described herein, for example those illustrated in FIGS. 1, 2 and 3, can also be used in a method of minimizing exposure of a subject to topiramate. The subject can be, for example, a subject who has taken an initial daily dose of topiramate for about two weeks and a second daily dose of topiramate for about three months according to the dosage regimes described herein. The subject can, after taking the initial and second daily doses of topiramate, have a body weight that is about 97% or more of the initial subject body weight, that is, the subject can have lost about 3% or less of the initial subject body weight and is likely a non-responder. In such a case, the method can include halting the administration of topiramate in the subject and thereby avoid exposing the subject to topiramate beyond the initial treatment and mitigating the potential for side effects. Alternatively, the method can include increasing the daily dose of topiramate to a third daily dose of topiramate, and subsequently to a fourth daily dose of topiramate, according to the dosing regimens described herein. Another method of minimizing exposure to a subject can including halting administration of topiramate to a subject who has taken the initial, second, third, and fourth daily doses of topiramate in the dosing regimens described herein, but whose weight is about 95% or more of the initial subject weight, that is, whose weight has not decreased by about 5% or more.

Although the exemplary method for identification of non-responders discussed above is failure to achieve at least 3% or at least 5% weight loss from baseline, other methods for identifying non-responders may also be used. For example, likelihood that a subject will or will not respond to topiramate or topiramate/phentermine combination therapy may be determined by a subject's genotype at one or more polymorphisms. Polymorphisms that have been identified as being relevant to topiramate response include rs2304016 in the SCN2A gene where the A allele was found to be associated with non-responsiveness to the drug (Kwan et al., Pharmacogenet Genomics 18(11):989-998 (2008)). SNPS rs2306719 and rs4984241 in the CA12 gene were found to influence drug response (Mirza et al., Pharmacogenet Genomics 21(5):297-302 (2011). Ray et al. identified a SNP (r52832407) in intron 9 of the glutamate receptor GluR5 gene (GRIK1) that may be related to topiramate-induced side effect severity and serum levels of topiramate, Ray et al., Exp. Clin. Psychopharmacol. 17(2), 122-129 (2009).

The topiramate used in the dosing regimens and methods described herein can be administered in any suitable dosage form, depending on the desired route of administration. For example, tablets, capsules, caplets, elixirs, syrups, sachets, granules, powders, pellets, and beads are all suitable for oral administration. Examples of dosage form suitable for topical administration include ointments, creams, liquids, solutions, suspensions, and pastes. Injectable dosage forms that can be used in, for example, parenteral, intramuscular, intravenous, or interarterial administration, include, for example, one or more of liquids, suspensions, solutions, and depots. Rectal and vaginal suppositories are exemplary dosage forms for rectal and vaginal administration, respectively. Dosage forms for these and other routes and modes of administration are discussed, for example, in Remington: The Science and Practice of Pharmacy, which is hereby incorporated by reference in its entirety.

Topiramate can be present in a controlled release dosage form, such as a sustained release form, a delayed release form, or a dosage form with both delayed and sustained release. Controlled release forms can be any controlled release form, and can be prepared by any preparation method known in the art. Some controlled release forms include topiramate dispersed within a matrix of one or more controlled release polymers, for example, one or more hydrolyzable or degradable polymers, such as one or more hydrophilic polymers. Other controlled release forms include a topiramate containing dosage form coated with one or more controlled release polymers. Exemplary hydrophilic polymers useful for this purpose include cellulose polymers, such as one or more of hydroxypropyl cellulose, hydroxyethyl cellulose, hydroxypropyl methyl cellulose, methyl cellulose (METHOCEL®), ethyl cellulose, cellulose acetate, cellulose acetate phthalate, and sodium carboxymethylcellulose, acrylic polymers and copolymers, such as polymers or copolymers of one or more of (meth)acrylic acid, methyl(meth) acrylate, and ethyl (meth)acrylate, vinyl polymers and copolymers, such polymers with one or more of polyvinyl pyrrolidone (POVIDONE® and POVIDONE® K30), polyvinyl acetate, and ethylene vinyl acetate.

Controlled release dosage forms, such as sustained release dosage forms, can also include additional excipients such one or more binders, diluents, bulking agents, glidents, lubricant, taste-modifying agents, flavorings, colorings, and the like. Such agents can be useful in the manufacturing process of the controlled release dosage form, commercially beneficial, for example, to provide a commercially desirable appearance, taste, or both. Many examples of such excipients are known in the art, and are discussed, for example, in Remington: The Science and Practice of Pharmacy, which is hereby incorporated by reference in its entirety.

Specific examples of controlled release dosage forms of topiramate include polymer matrices that contain the topiramate and a controlled release polymer, tablets coated with a controlled release polymer, osmotic tablets, and polymer coated beads. Such dosage forms can be prepared by methods known in the art, for example, methods described in U.S. Pat. No. 8,580,299, which is hereby incorporated by reference.

In an exemplary dosage form, the topiramate is present in a controlled release polymer coated bead. The polymer coated bead can contain a matrix bead core with topiramate and one or excipients, and a polymer coating. The one or more excipients in the matrix core can include one or more of microcrystalline cellulose and methylcellulose. The polymer coating can contain one or more controlled release polymers and one or more additional polymers, for example, ethyl cellulose and polyvinylpyrrolidone (POVIDONE™), such as POVIDONE™ K30. One or more polymer coated beads can be, for example, combined with tableting excipients, such as one or more binders, lubricants, glidant, etc., and compressed into one or more tablets. One or more polymer coated beads can also be prepared as one or more capsules, for example, by filling one or more capsule shells, such as gelatin capsule shells, with the one or more polymer coated beads.

When one or more sympathomimetic agents, such as phentermine, are administered with the topiramate, the one or more sympathomimetic agents can be present in the same dosage form as the topiramate or in a different dosage form. When the one or more sympathomimetic agents are in a different dosage form from the topiramate, the type of dosage form used for the one or more sympathomimetic agents can be the same or different from the type of dosage form used for the topiramate. For example, topiramate can be present in a capsule and phentermine can be present in a solution. In that example, the topiramate can be administered orally and the phentermine can be administered intra-muscularly. As another example, the topiramate and phentermine can be present in the same dosage form, such as a powder, bead, or granule, or in the same unit dosage form, such as a capsule, or tablet. When present in a tablet form the tablet can be a multilayer table, for example, a bilayer tablet having an immediate release portion containing the phentermine and a sustained release portion containing the topiramate. A tablet-in-tablet formulation can also be used, where the core comprises a therapeutically effective amount of topiramate that is surrounded by a layer comprising a therapeutically effective amount of phentermine. The topiramate and phentermine can be in direct contact of may be separated by a barrier layer. The core can contain both topiramate and one or more pharmaceutically acceptable excipients. The tablet can be coated with a rapidly dissolving coating or film.

The one or more sympathomimetic agents, such as phentermine, can be administered in an immediate release form dosage form. An exemplary immediate release form is an inert bead, such as a non-pareil or sugar sphere, coated with the one or more sympathomimetic agents, such as phentermine, to form one or more sympathomimetic agent coated beads. Additional coating agents, such as film-formers, diluents, plasticizers, binders, coating aids, adhesion aids, and the like, can also be present in the coating of the one or more sympathomimetic agent coated beads. Further, additional coating layers, such as film-coats or topcoats, can be present either on top of the sympathomimetic coating or between the inert bead and the sympathomimetic coating. One or more sympathomimetic agent coated beads can be, for example, mixed with one or more tableting excipients, such as one or more binders, lubricants, glidant, etc., and compressed into one or more tablets. One or more sympathomimetic agent coated beads can also be prepared as one or more capsules, for example, by filling one or more capsule shells, such as gelatin capsule shells, with the sympathomimetic agent coated beads.

When one or more sympathomimetic agents, such as phentermine, are in the same dosage form as the topiramate, such as a unit dosage form with topiramate and phentermine, the unit dosage form can contain a controlled release portion of topiramate and an immediate release portion of phentermine. For example, one or more polymer coated beads containing topiramate and one or more sympathomimetic agent coated beads can be present in the same dosage form.

One or more tablets can be formed from appropriate amounts of one or more polymer coated beads containing topiramate and one or more sympathomimetic agent coated beads can be mixed with one or more tableting excipients, such as the tableting excipients described above, and compressed into tablets. Similarly, one or more capsules can be formed from appropriate amounts of one or more polymer coated beads containing topiramate and one or more sympathomimetic agent coated beads can be prepared as one or more capsules, for example by filling one or more capsule shells, such as gelatin capsule shells, with the one or more polymer coated beads containing topiramate and one or more sympathomimetic agent coated beads. Compositions of this type can be prepared, for example, by the general methodology described in U.S. Pat. No.8,580,299, which is hereby incorporated by reference.

Polymer coated beads containing topiramate can contain a filler, such as microcrystalline cellulose, for example, in an amount of about 40% (w/w) to about 60% (w/w) or about 52% (w/w); a binder, such as methylcellulose, for example in an amount of about 1% to about 5% or about 3% (w/w); and can be coated with a sustained release polymer, such as ethylcellulose or ethylcellulose, in an amount of about 2% (w/w) to about 10% (w/w) or about 5% (w/w) or 5.5% (w/w), in combination with polyvinylpyrrolidone (POVIDONE™), in an amount of about 1% (w/w) to about 5% (w/w) or about 2.5% (w/w) or 2.4% (w/w). The topiramate beads can have a topiramate content of about 25% (w/w) to about 50% (w/w), such as about 29.25% (w/w) to about 46.25% (w/w), or about 36.84% (w/w). Topiramate can be the only active agent in the topiramate beads. Topiramate beads can be blended with about 0.1% (w/w) to about 2% (w/w), such as about 0.5% (w/w) of a glidant, which can be talc, to produce a topiramate bead glidant blend, such as a topiramate bead talc blend. The addition of a glidant, such as talc, can improve flow properties of the topiramate beads during the encapsulation process.

Beads containing a sympathomimetic agent, particularly phentermine, can have an inert core, such as a sugar sphere or non-pareil. The inert core can be coated with phentermine, such as phentermine hydrochloride, optionally in combination with a binder such as hydroxypropyl methylcellulose (sometimes known as Hypromellose or HPMC). The phentermine beads can have two different phentermine hydrochloride loading levels, for example, a first phentermine bead can have a phentermine loading of about 3.0% (w/w) to about 6.5% (w/w), such as about 3.75% (w/w) to about 5.85% (w/w), or 4.67% (w/w). At these loading levels, the first phentermine bead can have a hydroxypropyl methylcellulose content from about 2% (w/w) to about 8% (w/w), such as about 5% (w/w) or about 4.67% (w/w). A second phentermine bead can have a phentermine loading of about 6.5% (w/w) to about 13.0% (w/w), such as about 7.50% (w/w) to about 11.25% (w/w), or about 9.33% (w/w), to facilitate the different doses. At these loading levels, the second phentermine bead can have a binder, such as hydroxypropyl methylcellulose, content from about 4% to about 15%, such as about 10% or about 9.33%. The first or second phentermine beads, or both, can also have a ratio of phentermine to binder, such as hydroxypropyl methylcellulose, that is about 1:1. Phentermine can be the only active agent in the first phentermine bead, the second phentermine bead, or both. The first phentermine bead, such as a phentermine bead with about 4.67% phentermine loading, can be used to fill the lower dose capsules and the second phentermine bead, such as a phentermine bead with about 9.33% phentermine loading can be used to fill the higher dose capsules, for example, as shown in Table 3.

To obtain capsules of different dosages of phentermine and topiramate each capsule can contain a first amount of phentermine beads and a second amount of topiramate beads in the amounts required to achieve the desired dosage strengths of phentermine and topiramate. For example, bead mixtures with about 23 mg of topiramate and 3.75 mg phentarmine can contain about 80 mg to about 125 mg, such as about 100 mg, of first phentermine beads with about 50 mg to about 79 mg, such as about 63 mg, of topiramate bead glidant blend. Bead mixtures with about 46 mg of topiramate and about 7.5 mg of phentermine can contain about 160 mg to about 250 mg, such as about 200 mg, of first phentermine beads with about 100 mg to about 158 mg, such as about 126 mg, of topiramate glidant blend. Bead mixtures with about 69 mg of topiramate and about 11.25 mg of phentermine can contain about 120 mg to about 188 mg, such as about 150 mg, of second phentermine beads and about 150 mg to about 235 mg, or about 188 mg, of topiramate bead glidant blend. Bead mixtures with about 92 mg of topiramate and about 15 mg of phentermine can contain about 160 mg to about 250 mg, such as about 200 mg, of second phentermine beads and about 200 mg to about 315 mg, such as about 251 mg, of topiramate glidant blend. Table 3 shows a composition of exemplary phentermine and topiramate containing capsules at each of the four dosage strengths discussed above.

TABLE 3

| | 3.75/23 | | 7.5/46 | 11.25/69 | 15/92 |
|---|---|---|---|---|---|
| Component | mg | | mg | mg | mg |
| Phentermine Beads | 100 | mg | 200 mg | Not used | Not used |
| 4.67% loading | | | | | |
| Phentermine Beads | Not used | | Not used | 150 mg | 200 mg |
| 9.33% loading | | | | | |
| Topiramate Bead Talc | 63 | mg | 126 mg | 188 mg | 251 mg |
| Blend | | | | | |

| | | | | |
|---|---|---|---|---|
| Printed Hard Gelatin | 1 | 1 | 1 | 1 |
| Capsule | | | | |

Particular compositions of the individual components of the phentermine beads, which are not intended to be limiting unless otherwise specified, are shown in Table 4. Particular compositions of topiramate beads and topiramate bead talc blend, which are likewise not intended to be limiting unless otherwise specified, are shown in Table 5. Water and denatured alcohol can be used as processing aids and can be removed during the manufacturing process so that only a trace amount, or no measureable amount, of either water or denatured alcohol is present in the final product.

TABLE 4

| | | PHEN Beads | |
|---|---|---|---|
| | | 4.67% loading | 9.33% loading |
| Component | Function | level % (w/w) | level % (w/w) |
| Phentermine | Active | 4.67 | 9.33 |
| Hydrochloride, USP | Ingredient | | |
| Sugar Spheres 20/25 | Substrate | 90.66 | 81.34 |
| mesh, NF | | | |
| Hypromellose 2910, | Binder | 4.67 | 9.33 |
| USP | | | |

| | | |
|---|---|---|
| Purified Water, USP | Solvent | Not applicable |

| | | |
|---|---|---|
| Total | 100.00 | 100.00 |

TABLE 5

| | | | TPM Beads |
|---|---|---|---|
| | | TPM Beads | Talc Blend |
| Component | Function | % (w/w) | % (w/w) |
| Topiramate, USP | Active Ingredient | 36.84 | 36.65 |
| Microcrystalline | Filler | 52.03 | 51.77 |
| Cellulose, NF | | | |
| Methylcellulose, USP | Binder | 3.22 | 3.21 |
| Ethylcellulose, NF | Coating component | 5.51 | 5.49 |
| Povidone K 30, USP | Coating component | 2.40 | 2.38 |
| Micronized Talc, NF | Lubricant | 0 | 0.5 |

| | | |
|---|---|---|
| Denatured ethanol | Solvent for coating | Not applicable |
| | of TPM Beads | |
| Purified Water, USP | Solvent for | Not applicable |
| | manufacture of core | |
| | beads | |

| | | |
|---|---|---|
| Total | 100.00 | 100.00 |

Any of the capsules, mixtures of beads, or dosage forms, discussed above can be used in any of the methods or dosing regimens discussed herein.

One or more of the dosing regimens, methods, and dosage forms described herein can be used to treat a variety of conditions, including obesity, morbid obesity, excess weight, as well as the various conditions that are associated with obesity, morbid obesity, or excess weight. Thus, subjects suitable for treatment by the dosing regimens and methods described herein include, but are not limited to, those suffering from or at risk for suffering from one or more of the following conditions: diabetes, insulin resistance, impaired glucose tolerance; respiratory problems such as pulmonary hypertension, asthma, and shortness of breath; gallbladder disease; dyslipidemia, high levels of triglycerides, and hypercholesteremia; osteoarthritis and other orthopedic problems; reflux esophagitis; sleep apnea and loud snoring; menstrual irregularities, infertility, and complications during pregnancy; gout; hypertension; coronary artery disease, chronic heart disease, congestive heart failure, and acute heart failure; muscular dystrophy; stroke, thrombotic stroke, and deep vein thrombosis; migraines; metabolic disorders such as hypoalphalipoproteinemia, familial combined hyerlipidemia, Syndrome X and insulin-resistant Syndrome X; psychiatric conditions including depression, such as major depression, refractory depression, bipolar depression, impulse control disorders, general anxiety disorders; and cancer, such as cancer of the colon, rectum, liver, esophagus, gallbladder, pancreas, prostate, breast, uterus, ovaries, endometrium, and cervix.

The above-mentioned indications can be associated with or caused by obesity, morbid obesity, or excess weight, and as such can be treated by causing weight loss. The relationship between these and other conditions and obesity, morbid obesity, or excess weight has been documented in the art, and is discussed, for example, in U.S. Pat. Nos. 7,056,890, 8,580,298, and 8,580,299, which are hereby incorporated by reference.

Complications associated with diabetes, including heart disease and stroke, high blood pressure, blindness, kidney disease, dental disease, complications of pregnancy, neuropathy and amputation, may also be mitigated.

One or more dosage forms of topiramate, for example, for use in one or more of the dosing regimens or methods described herein, such as the dosage forms described herein, can be packaged into a convenient packaging for delivery to or use by one or more physicians, subjects, nurses, health-care professionals, etc. Such packaging can include one or more sealed containers, each containing one or more dosage forms of topiramate, such as the dosage forms described herein.

Prepackaged pharmaceutical preparations can include, for example, an appropriate number of dosage forms, for example capsules as described in Table 3, for administration to a subject for all or a portion of the dosing regimens or methods described herein. For example, when topiramate is to be administered according to the dosing regimen of FIG. 1, a prepackaged pharmaceutical preparation can include about two weeks a daily dose of about 23 mg of topiramate and about 3 months of a daily dose of about 46 mg of topiramate. A prepackaged pharmaceutical preparation can also include about two weeks of a daily dose of about 69 mg of topiramate and about three months of a daily dose of about 92 mg of topiramate. As another example, when a combination of topiramate and phentermine are to be administered according to the dosing regimen of FIG. 2, a prepackaged pharmaceutical preparation can include about two weeks a daily dose of about 23 mg of topiramate with 3.75 mg of phentermine and about 3 months of a daily dose of about 46 mg of topiramate with 7.5 mg phentermine. A prepackaged pharmaceutical preparation can also include about two weeks of a daily dose of about 69 mg of topiramate with about 11.25 mg phentermine and about three months of a daily dose of about 92 mg of topiramate with about 15 mg phentermine.

While various embodiments have been described in detail in order to explain the invention, such embodiments are not intended to be limiting unless otherwise specified. Indeed, a person of skill in the art will recognize that modifications, additions, and substitutions can be implemented without altering the scope or spirit of the invention. For example, while embodiments featuring one or more sympathomimetic agents have been described with reference to phentermine and particular dosages thereof, other sympathomimetic agents can be used in appropriate dosages to achieve similar results.

1. A method of administering topiramate to a subject comprising
measuring the weight of a subject to obtain a initial subject weight;
administering a daily dose of about 23 mg of topiramate to the subject for about two weeks after measuring the weight of the subject;
administering a daily dose of about 46 mg of topiramate to the subject for about three months after the about two weeks of administering a daily dose of about 23 mg topiramate;
measuring the weight of the subject after the about three months of administering a daily dose of about 46 mg of topiramate to determine a first weight change of the subject from the initial subject weight; and wherein
if the weight of the subject has decreased by about 3% or more, maintaining administration of the daily dose of about 46 mg of topiramate; or wherein
if the weight of the subject has not decreased by about 3% or more, administering a daily dose of about 69 mg of topiramate.

2. The method of claim 1, wherein
administering a daily dose of about 69 mg of topiramate comprises administering the daily dose of about 69 mg of topiramate for about two weeks after determining the first weight change.

3. The method of claim 2, further comprising
administering a daily dose of about 92 mg of topiramate to the subject for about three months after the about two weeks of administering the daily dose of about 69 mg of topiramate.

4. The method of claim 2, further comprising
measuring the weight of a subject after the about three months of administering the 92 mg of topiramate to determine a second weight change of the subject from the initial subject weight; and wherein
if the weight of the subject has decreased by about 5% or more, maintaining administration of the daily dose of about 92 mg of topiramate; or wherein
if the weight of the subject has not decreased by about 5% or more, halting the administration of topiramate.

5. The method of claim 1, further comprising,
administering a daily dose of a sympathomimetic agent with the daily dose of about 23 mg of topiramate.

6. The method of claim 5, wherein the sympathomimetic agent is phentermine.

7. The method of claim 6, further comprising
administering a daily dose of about 3.75 mg of phentermine with the daily dose of about 23 mg of topiramate.

8. The method of claim 7, wherein administering a daily dose of about 3.75 mg of phentermine with the daily dose of about 23 mg of topiramate further comprises
administering a controlled release form of the about 23 mg of topiramate; and
administering an immediate release form of the about 3.75 mg of phentermine.

9. The method of claim 8, wherein
the controlled release form of the about 23 mg topiramate and the immediate release form of the about 3.75 mg of phentermine constitute a single dosage form.

10. The method of claim 9, wherein the single dosage form is a capsule comprising polymer coated beads or a bilayer tablet.

11. The method of claim 1, further comprising
administering a daily dose of a sympathomimetic agent with the daily dose of about 46 mg of topiramate.

12. The method of claim 11, wherein the sympathomimetic agent is phentermine.

13. The method of claim 12, further comprising administering a daily dose of about 7.5 mg of phentermine with the daily dose of about 46 mg of topiramate.

14. The method of claim 13, further comprising
administering a daily dose of about 3.75 mg of phentermine with the daily dose of about 23 mg of topiramate.

15. The method of claim 14, wherein
administering a daily dose of about 3.75 mg of phentermine with the daily dose of about 23 mg of topiramate further comprises administering a controlled release form of the about 23 mg of topiramate, and administering an immediate release form of the about 3.75 mg phentermine; and wherein
administering a daily dose of about 7.5 mg of phentermine with the daily dose of about 46 mg of topiramate further comprises administering a controlled release form of the about 46 mg topiramate; and administering an immediate release form of the about 7.5 mg of phentermine.

16-31. (canceled)

32. A method of administering topiramate to a subject who
has an initial body weight prior to taking topiramate;
has taken a daily dose of about 23 mg of topiramate for about two weeks; and
has taken a daily dose of about 46 mg of topiramate for about three months after the about two weeks of taking the about 23 mg of topiramate; and
has a body weight after the about three months of taking the daily dose of about 46 mg of topiramate that is about 97% or more of the initial body weight;
the method comprising administering to the subject a daily dose of about 69 mg of topiramate for about two weeks or halting administration of topiramate to the subject.

33-73. (canceled)

74. A method of minimizing exposure to topiramate in a subject who
has an initial body weight prior to taking topiramate;
has taken a daily dose of about 23 mg of topiramate for about two weeks; and
has taken a daily dose of about 46 mg of topiramate for about three months after the about two weeks of taking the about 23 mg of topiramate;
has taken a daily dose of about 69 mg of topiramate for about two weeks after the about three months of taking a daily dose of about;
has taken a daily dose of about 92 mg of topiramate for about three months after the about two weeks of taking a daily dose of about 69 mg of topiramate; and
has a body weight after the about three months of taking a daily dose of 92 mg topiramate that is about 95% or more of the initial body weight;
the method comprising halting administration of topiramate to the subject.

75-100. (canceled)

101. A composition comprising:
(i) one or more sustained release topiramate beads, the sustained release topiramate beads consisting essentially of
about 1% (w/w) to about 5% (w/w) of a binder;
about 40% (w/w) to about 60% (w/w) of a filler;
about 25% (w/w) to about 50% (w/w) of an active agent, the active agent consisting of topiramate; and
a sustained release polymer coating; and either
(ii) one or more first phentermine beads, the first phentermine beads consisting essentially of
an inert core;
about 3% (w/w) to about 6% (w/w) phentermine on the inert core; and
about 2% to about 8% of a binder coated on the inert core; or
(iii) one or more second phentermine beads, the second phentermine beads consisting essentially of
an inert core;
about 6.5% (w/w) to about 13% (w/w) phentermine coated on the inert core; and
about 4% to about 15% of a binder coated on the inert core.

102-132. (canceled)
